(19) United States
(12) Patent Application Publication
(10) Pub. No.: US 2026/0209659 A1
(43) Pub. Date: Jul. 23, 2026

(54) TEMPORAL VARIATIONS IN FLUX OUTPUT

(71) Applicant: University of Technology Sydney, Ultimo, New South Wales (AU)

(72) Inventors: Andrei Herdean, Ultimo (AU); Bernardo Campos Diocaretz, Ultimo (AU); Peter RALPH, Ultimo (AU)

(21) Appl. No.: 19/139,925

(22) PCT Filed: Dec. 18, 2023
(86) PCT No.: PCT/AU2023/051318

§ 371 Date: Jun. 17, 2025

(30) Foreign Application Priority Data
Dec. 19, 2022 (AU) 2022903903

(51) Int. Cl. C12M 1/00; C12M 1/12; C12M 1/32; C12M 1/36; C12M 3/00; C12N 1/12; C12R 1/89

(52) U.S. Cl. CPC C12M 21/02; C12M 23/12; C12M 23/44; C12M 25/06; C12M 41/06; C12M 41/48; C12N 1/12; C12R 2001/89

(57) ABSTRACT

A device for assessing temporal variations in flux output. The device includes at least one light source, a sample plate; at least one sensor and a processor. The light source can emit light in accordance with temporally varying flux output conditions. The sample plate is affixed above the light source and configured to receive at least one sample. The processor is in communication with the light source and configured to control the light source to provide a flux output, the flux output including at least a duty cycle and a pulse frequency. The sensor is configured to obtain a measurement of at least one response parameter of the sample. The processor is also configured to control the light source to adjust the provided flux output based on the at least one response parameter.

### REFERENCE TO RELATED APPLICATIONS

This application claims Convention priority from Australia Provisional Patent No. 2022903903, filed 19 Dec. 2022, the contents of which are incorporated herein by reference.

### FIELD OF THE INVENTION

The present invention relates to temporal variations in flux output. More particularly, the invention relates to systems, devices and methods for determining optimised temporal variations in flux output for use in algae cultivation.

The invention has been developed primarily for use as a testing device to determine optimised temporal variations in flux output for use in photobioreactors, and in particular for cultivation of algae in photobioreactors. However, while some embodiments will be described herein with particular reference to that application, it will be appreciated that the invention is not limited to such a field of use, and is applicable in broader contexts.

### BACKGROUND

The following discussion of the prior art is intended to facilitate an understanding of the invention and to enable the advantages of it to be more fully understood. It should be appreciated, however, that any reference to prior art throughout the specification in no way be considered as an admission that such art is widely known or forms part of the common general knowledge in the field.

Changes in frequency, intensity and duty cycle of flux, or flickering illumination, has been known to impact the photosynthesis process of plants. The productivity of algae (including cyanobacteria) has been shown to increase and decrease when exposed to flickering of light. Over the years, inconsistent observations of the impact of temporal variations in flux have been widespread. The impact of flickering illumination conditions is likely species-specific and possibly influenced by the growth condition of the algae. Research groups globally have manually trialed a vast array of frequencies, amplitudes and duty cycles, but not in a systematic manner and not with sufficient resolution to know that an especially promising “sweet spot” for a particular plant or algae species has not been overlooked. That is, the existing method for determining the best temporal variation in flux output to which plants and algae should be exposed is largely a trial-and-error approach. Given the immense number of possible permutations of frequency and duty cycle, the trial-and-error manual approach is impractical to use to determine the optimal temporal variations for illumination of flux output.

It is an object of the present invention to overcome or ameliorate at least one of the disadvantages of the prior art, or to provide a useful alternative.

### SUMMARY OF THE INVENTION

According to a first aspect of the invention, there is provided a device for assessing temporal variations in flux output, including:
at least one light source able to emit light in accordance with temporally varying flux output conditions;
a sample plate affixed above the at least one light source, configured to receive at least one sample;
a processor in communication with the light source and configured to control the at least one light source to provide a flux output, the flux output including at least a duty cycle and a pulse frequency;
at least one sensor configured to obtain a measurement of at least one response parameter of the at least one sample; and
wherein the processor is further configured to control the at least one light source to adjust the provided flux output based on the at least one response parameter.

Preferably, the at least one light source and the sample plate are disposed on a modular base.

In one embodiment, the at least one light source is a light-emitting element. Preferably, the at least one light source is an array of light-emitting elements. In a further embodiment, the flux output further includes intensity.

In another embodiment, the sample is algae. Preferably, the algae is a microalgae.

The duty cycle and the pulse frequency are preferably adjusted to enable temporal variation of the flux output over a defined time interval.

In one embodiment, the sample plate is a microplate. In a further embodiment, the microplate is a single well microplate. In another embodiment, the microplate includes an array of equally spaced wells. Preferably, the microplate is a 96-well microplate.

Preferably, the at least one sensor is a CCD camera. More preferably, the CCD camera is configured to perform a fluorometric assessment. The camera is preferably fitted with a Chlorophyll a specific emission filter.

According to a second aspect of the invention, there is provided a method for determining optimal temporal variations in flux output for algal growth, including the steps of:
providing at least one sample on a sample plate;
controlling at least one light source to emit light towards the sample plate in accordance with flux output conditions, wherein the flux output conditions include at least a duty cycle and a pulse frequency;
exposing the sample to the emitted light for a predefined time interval;
obtaining a first measurement of at least one response parameter related to the at least one sample after it has been exposed to the emitted light for a defined time interval;
adjusting the flux output conditions based on the at least one response parameter;
obtaining a second measurement of at least one response parameter related to the at least one sample after it has been exposed to emitted light having adjusted flux output conditions for a second defined time interval;
identifying an optimal set of flux output conditions from the at least one response parameter when the response parameter is determined to reach a threshold value; and
outputting the optimal set of flux output conditions as an optimal temporal variation of flux output for the sample.

Preferably, the first measurement or the second measurement is obtained by at least one sensor. The at least one response parameter preferably includes the results of a fluorometric assessment.

In one embodiment, adjusting the flux output conditions includes adjusting at least one of the duty cycle or the pulse frequency. In a further embodiment, identifying an optimal set of flux output conditions from the at least one response parameter includes comparing the first measurement and the second measurement. In yet another embodiment, identifying an optimal set of flux output conditions from the at least one response parameter incudes comparing the first measurement and the second measurement with historical measurements.

### BRIEF DESCRIPTION OF THE DRAWINGS

Preferred embodiments of the disclosure will now be described, by way of example only, with reference to the accompanying drawings in which:

FIG. 1 is an image of a top view of the assessment device according to one embodiment of the invention;

FIG. 2 is an image of a side view of the assessment device shown in FIG. 1;

FIG. 3 is a circuit diagram of the electronics in the assessment device according to one embodiment of the invention;

FIG. 4 is a circuit diagram of the electronics in the assessment device according to one embodiment of the invention;

FIG. 5 is a diagram of the closed loop feedback system according to one embodiment of the method of the invention.

FIG. 6 is a table showing the scan of frequency and duty cycles on a sample of Chlorella vulgaris using one embodiment of the device;

FIG. 7A is graph showing the results of different flux output conditions tested on a sample of Scenedesmus in accordance with one embodiment of the method; and

FIG. 7B is graph showing the results of different flux output conditions tested on a sample of Chlorella vulgaris in accordance with one embodiment of the method.

### DETAILED DESCRIPTION

### Device Overview

There are provided systems and devices for determining optimised temporal variations in flux output. There are also provided systems and devices for providing the optimised temporal variations in flux output to a photobioreactor. It will be appreciated that the term “flux” used herein may include luminous flux, radiant flux or both.

The present invention relates to an assessment device that is able to expose samples of microalgae or other photosynthetic organisms to a wide variety of combinations of frequency, intensity and duty cycle; with the specific intention of identifying the optimal conditions for max biomass production or max cellular component (such as pigment/cell or metabolite/cell). One embodiment of the assessment device is based on a 96-well plate with an array of LEDs controlled by a series of microchips driving the illumination of each well. Raspberry pi control units allow full manipulation of the frequency, intensity and duty cycle. Each test runs for at least 10 minutes of exposure followed by a fluorometric assessment of the impact via a CCD camera fitted with a chlorophyll a specific emission filter. The device consisting of the 96 LED array is modular and designed to couple together with multiple units for performing algae growth tests in hundreds of different flickering illumination conditions.

There is also provided a method of determining the optimal temporal variation of flux output from the LEDs for each sample. This involves exposing the samples to light for about 10 minutes, performing an assessment of how the sample responds to the light conditions (by monitoring a particular response parameter), and then adjusting the flux output conditions by manipulating at least one of the pulse frequency, duty cycle or intensity. The sample is then exposed again, and further measurements are taken to determine how the sample is responding in comparison with the first exposure. The method iteratively adjusts the flux output conditions until a response parameter of the sample falls within a predefined range or above a threshold which defines optimisation. The identified flux output conditions are then output as the optimal temporal variations in illumination.

### Device (Including Sample Plate, Controller, LEDs, Sensor)

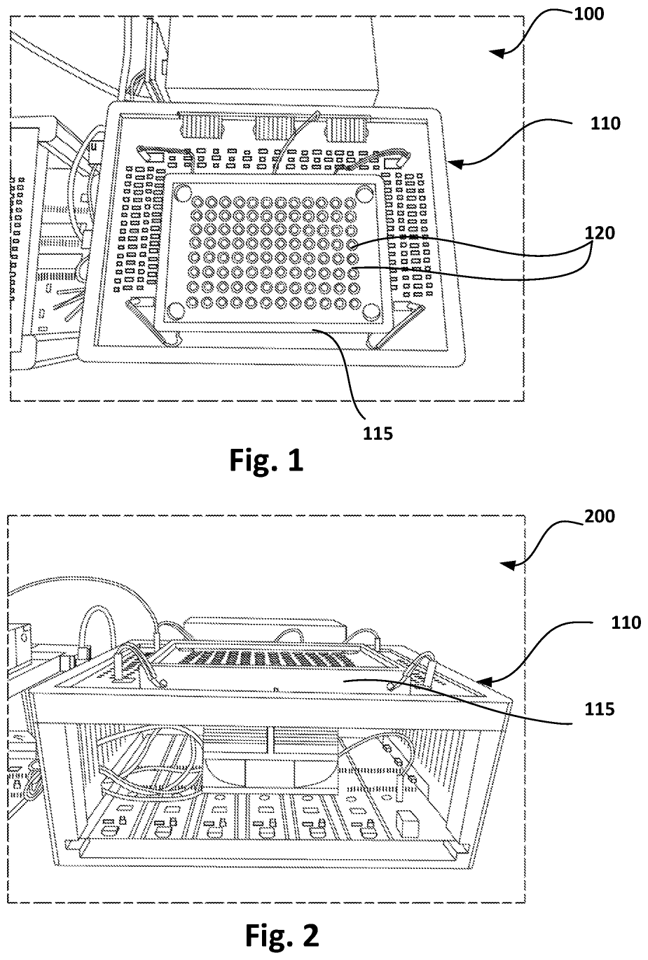

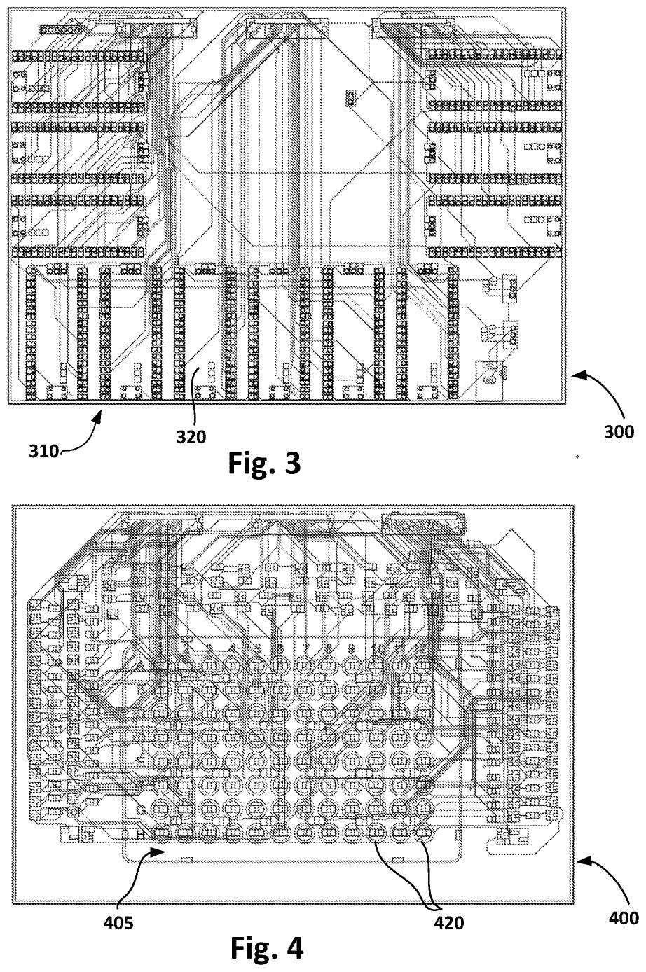

In overview, there is provided an assessment device for determining optimal temporal variations in flux output for algal growth. In other embodiments, the assessment device may determine optimal temporal variations in flux output for product or cell growth. Flux output may also be referred herein as “illumination”. Temporal variations in illumination can have a large effect on growth of algae. In a preferred embodiment, the assessment device includes at least one light source, a sample plate affixed above the at least one light source, a processor configured to control the light source to provide a flux output, and at least one sensor. FIG. 1 shows a top view 100 of an example assessment device 110. FIG. 2 shows a side view 200 of the assessment device 110. FIG. 3 shows an example view 300 of electronics of the assessment device 110. FIG. 4 shown a second example view 400 of electronics of the assessment device 110.

The device 110 includes a sample plate 115 configured to receive or contain a sample. The sample plate 115 may be a microplate. In the embodiments shown in FIGS. 1 and 2, the sample plate 115 is a microplate. The microplate 115 is affixed above the at least one light source, configured to receive at least one sample. In some embodiments, the microplate is a single-well microplate. The single-well microplate may be in the form of a tray or receptacle for holding samples. In further embodiments, the microplate includes a plurality of wells (for example, wells 120). The plurality of wells may be arranged in an array, and may be equally spaced apart from each other. The microplate 115 may be a 96 well microplate. The 96 well microplate is arranged in a 12 by 8 formation, with the center of each of the wells (such as 120) being spaced about 9 mm apart. The 96-well microplate is arranged to sit directly above an array of 96 LEDs, with each LED corresponding to a single well. The wells (for example, 120) of the microplate may be between about 2 mm and about 15 mm in depth, and particularly between about 6 mm and about 13 mm, and more particularly between about 10 mm and about 12 mm. The wells of the microplate may be between about 4 mm and about 10 mm in diameter, and particularly between about 6 mm and about 8 mm in diameter. Preferably, the wells of the microplate are approximately about 4.2 mm in depth and about 2.7 mm in diameter, and holding a culture volume of about 200 μL. The wells of the microplate may have a U-shaped, V-shaped, F-shaped, or C-shaped base.

Preferably, the sample provided in the wells of the microplate 115 includes algae. The algae may be a microalgae. Alternatively, the algae may be a cyanobacteria. Alternatively, the sample may include other photosynthetic material. While the embodiments herein reference algae, it will be appreciated that the method and system can be applied to various plants, plant materials, protoplasts, bacteria and archaea, and that the devices, systems and methods can be used in various applications including, but not limited to, horticultural, agricultural, and aquacultural environments, as well as commercial glasshouses, hydroponics, tank-based seaweed, and vertical farming production.

The assessment device 110 includes at least one light source (for example, light source 405 in FIG. 4) able to emit light in accordance with temporally varying flux output conditions. That is, the light source 405 is able to provide illumination to a sample with a specific “flickering” pattern. The light source 405 may include a light-emitting element (LEE). A light emitting element may be any device that emits electromagnetic radiation at a defined wavelength. For example, visible, infrared or ultraviolet wavelengths. The emitted light is preferably in the visible spectrum, but may also include infrared or ultraviolet wavelengths. The light emitting elements may be activated by passing a current through the element, or applying a potential difference across the element. The light-emitting elements may include a semiconductor device. The light-emitting element may include, but is not limited to, solid-state, organic, polymer, phosphor-coated or high-flux LEDs, and/or laser diodes.

The light source 405 is a light-emitting element including at least one light emitting diode (LED) in some embodiments. The LED may be a LTW-150TK SMD white LED. In one embodiment, the LED is a full-spectrum SMD LED with dimensions of about 3.2 mm by about 1.6 mm. In other embodiments, the Blue LEDs which emit light in the range of about 460 nm to about 470 nm. The light source may be of any appropriate size to suit the assessment apparatus. In some embodiments, the LED may have at least one measurement/dimension less than 500 microns. Other sizes of LEDs may be of dimensions of about 200 microns by about 600 microns, about 250 microns by about 400 microns, about 250 microns by about 300 microns, or about 200 microns by about 200 microns. In some embodiments, where the light-emitting element includes a microLED, which may have at least one measurement/dimension less than about 300 microns. Other sizes of microLEDs may be of dimensions of about 200 microns or less than about 100 microns. For example, a microLED may have a size of about 200 microns by about 175 microns or about 150 microns by about 100 microns or about 150 microns by about 50 microns.

The light source may include an array of light-emitting elements. In a preferred embodiment, the light source is an array of light-emitting elements, where the light-emitting elements are a plurality of LEDs (for example LEDs 420 in FIG. 4). Where the light source 405 is an array of LEDs, the LEDs may be arranged in a rectangular array. Preferably the LEDs (for example 420) are equidistantly spaced apart from each other. The LEDs may be arranged in a 12 by 8 grid, so as to align with individual wells of a 96 well sample plate. However, it will be appreciated that the number of LEDs does not need to align with the number of wells on the microplate. In some embodiments, the number of LEDs provided is the same as the number of wells of the microplate. In other embodiments, the number of LEDs per well of a microplate may be more than one.

The LEDs are in communication with a processor (for example 310 of FIG. 3), which is configured to control the LEDs to provide illumination. The illumination or flux output provided by the LEDs includes at least a duty cycle and a pulse frequency. In further embodiments, the flux output also includes an intensity. The processor 310 may include a controller (for example 320), which is connected via electrical conductors to the LEDs, as shown in the circuit diagrams provided in FIGS. 3 and 4. The controller 320 controls the LEDs to provide illumination in accordance with particular flux output conditions. The processor 310 controls a drive current to be delivered to the LED, and may be controlled by analog circuity. In an alternative embodiment, the drive current may be controlled by digital circuitry, and involve digitally modulating a signal to the LED.

LEDs are well suited to deliver temporal variations in flux output or flickered illumination. Reducing the duty cycle of LEDs also reduces the electricity required to drive the illumination, therefore reducing the OPEX of producing biomass.

The processor 310 is able to set the flux output conditions, including a duty cycle, pulse frequency and intensity, of the emitted light, and provide temporally modulated drive current to the light source on the basis of these flux output conditions. The light source 405 then emits light towards at least one sample in the microplate 115 with illumination corresponding to those flux conditions. In some embodiments, the processor is configured to receive a signal in the form of a measurement obtained by at least one sensor. The processor 310 can then incorporate the obtained measurement and adjust the flux output, thereby creating a closed loop feedback system for self-adjusting the temporal variations in illumination in real time to provide optimised conditions. Additionally, the ability for the processor to control the drive current to the LEDs enables more efficient energy usage by avoiding continuous current and utilising intermittent current. For example, in testing of the device, on a sample of Scenedesmus sp, power was reduced by approximately 50% whilst promoting growth by 350%. Continuous current provided to the LED light sources (without flickering of the illumination) used 25.8 W over a period of about 4 hours. When temporal variations in the illumination were utilized, the power consumption reduced to 13.2 W.

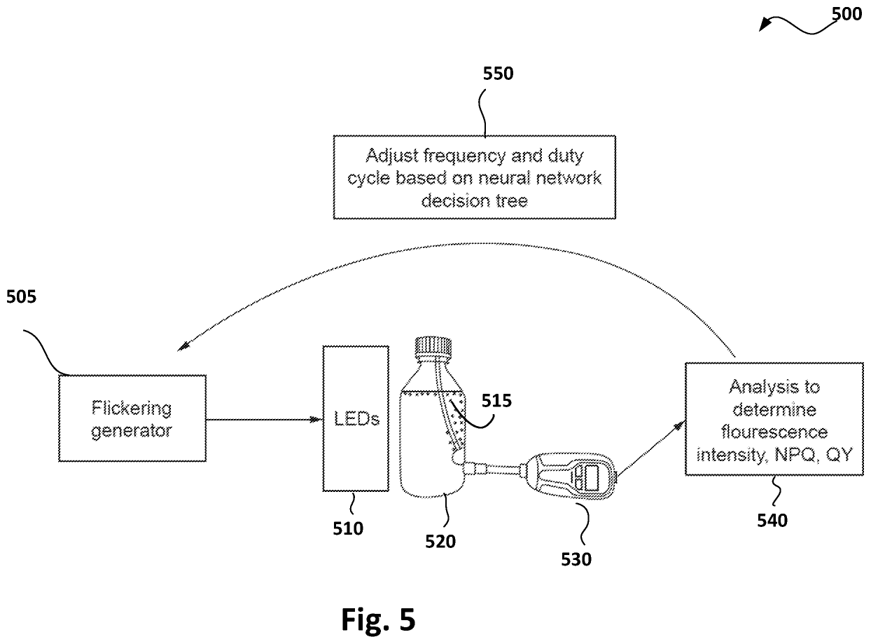

The assessment device 110 further includes at least one sensor (for example a sensor 530 of FIG. 5). The sensor is configured to obtain a measurement of at least one response parameter of the at least one sample. The sensor may include a measurement sensor for measuring physical growth of the sample, a temperature sensor, an infrared sensor, gas sensor, photorespiration sensor, near-infrared sensor, pH sensor, colorometric sensor, or emission sensor. For algae cultivation, the at least one sensor monitors one or more characteristics associated with the algae sample. For example, to determine the growth rate of the algae sample and determine whether the growth rate is increasing or stagnating under the provided illumination conditions.

The sensor is preferably a CCD camera. The CCD camera may be fitted with a chlorophyll a specific emission filter. Chlorophyll a fluorescence indicates whether the algae is healthy and is able to transform light into biological products. The emission filter enables a fluorometric assessment to be performed to ascertain the impact of the illumination conditions on the sample. In other embodiments, the camera may be fitted with other appropriate optical absorption or emission filters.

The response parameter may include any parameters or characteristics relating to the sample growth which are then used to optimise the provision of the temporal variations in illumination provided by the light source (such as 405). For example, the chlorophyll a fluorescence may be monitored with at least one sensor and form part of the response parameter to determine whether the illumination conditions are impacting the sample growth. That is, the response parameter may provide a measure of how the sample is responding to the illumination conditions. In another embodiment, the response parameter may include the optical density of the sample. The optical density of the sample measured between 670 nm and 750 nm can be used to determine whether the algae is growing better under particular flux output conditions. In another embodiment, a plate reader may be used with a dye to identify and measure lipid yield of the sample. For example, identifying lipids for reactive oxygen species. It will be appreciated that a number of other suitable measurements can be included in the response parameter. The response parameter can then be used to provide a basis for adjusting the duty cycle, pulse frequency or intensity of the flux output conditions to optimize the temporal variations for sample growth.

### System and Method for Optimization

There are also provided systems and methods for determining optimised temporal variations in flux output, and providing the optimised temporal variations in flux output within a photobioreactor.

In one embodiment, there is provided a method for determining optimal temporal variations in flux output for algal growth, including the steps of:
providing at least one sample on a sample plate;
controlling at least one light source to emit light towards the sample plate in accordance with flux output conditions, wherein the flux output conditions include at least a duty cycle and a pulse frequency;
exposing the sample to the emitted light for a predefined time interval;
obtaining a first measurement of at least one response parameter related to the at least one sample after it has been exposed to the emitted light for a defined time interval;
adjusting the flux output conditions based on the at least one response parameter;
obtaining a second measurement of at least one response parameter related to the at least one sample after it has been exposed to emitted light having adjusted flux output conditions for a defined time interval;
identifying an optimal set of flux output conditions from the at least one response parameter when the response parameter is determined to reach a threshold value; and
outputting the optimal set of flux output conditions as an optimal temporal variation of flux output for the sample.

A system for carrying out the above-described method is also provided. The system may include the assessment device 110 configured to carry out the above method. It may additionally include other components, such as a photobioreactor in communication with the assessment device 110, such that the system enables a closed-loop feedback system to continuously adjust the photobioreactor conditions to provide optimal temporal variations in illumination based on the response of the algae being cultivation.

In one embodiment, the sample used is algae. The algae samples are provided in the wells of the microplate. A light source (such as 405) in the form of an LED array is arranged beneath the wells of the microplate (such as 115). The LEDs are controlled to emit light towards the microplate in accordance with flux output conditions. The flux output conditions include at least a duty cycle and a pulse frequency. The sample of algae is exposed to the emitted light for a predefined time interval. The predefined time interval may be in the range of minutes to days. In one embodiment, the time interval is 10 minutes. After the predefined time interval has elapsed, a first measurement is obtained of a response parameter related to the sample. In one embodiment, a CCD camera fitted with a chlorophyll a specific emission filter which performs a fluorometric assessment to determine the chlorophyll emission and determine whether the sample is responding to the flux output conditions.

The flux output conditions are then adjusted based on the response parameter. In some embodiments, the duty cycle can be adjusted without adjusting the pulse frequency or the intensity, the pulse frequency can be adjusted without adjusting the duty cycle or the intensity, and the intensity can be adjusted without adjusting the duty cycle of the pulse frequency. A second measurement of the response parameter is obtained after the sample of algae has been exposed to emitted light having the adjusted flux output conditions for a second defined time interval. The second defined time interval may be the same as or different to the first defined time interval.

The method of monitoring the sample for a response parameter and then using the response parameter to adjust the flux output conditions of the emitted light from the light source (for example, 405) creates a closed loop feedback system, in which temporal variations of the flux output can be iteratively adjusted in real time in response to how the sample is responding to the provided illumination. In further embodiments, the method can monitor a sample of a photosynthetic organism throughout its lifecycle and determine optimal temporal variations for different stages of growth.

When the response parameter is determined to have reached a threshold value, an optimal set of flux output conditions is identified. The optimal set of flux output conditions is then output as an optimal temporal variation of flux output for the sample of algae. In other embodiments, instead of a threshold value, an upper and/or lower limit may be used to define a range within which the growth or response parameter is considered optimal. Identifying an optimal set of flux output conditions from the at least one response parameter may include comparing the first measurement and the second measurement. In other embodiments, identifying an optimal set of flux output conditions from the at least one response parameter incudes comparing the first measurement and the second measurement with historical measurements.

In further embodiments of the system, at least one response parameter may be continuously or intermittently monitored and the resultant measurements processed within a neural network control system which can learn optimum combinations of temporal variation and flux output conditions for different algae species, thereby predicting the optimized temporal variations on the basis of historical and observed trends for biological requirements. The at least one response parameter, and any other input data, can be collected and stored in a database, either locally or offsite. The collected data can then be used to improve the adjustments made by the system and method, or for additional study and analysis. An example embodiment of this system is shown in FIG. 5.

In an example system 500 of FIG. 5, a flickering generator 505 controls operation of a set of LEDs 510. The LEDs 510 emit light onto a container 520 which contains an algal solution 515. A sample of the solution 515 is provided to the sensor 530. The example sensor 530 comprises a fluorometric sensor. The sensor 530 outputs fluorescence measurements to an analysis module 540. The analysis module 540 operates to determine fluorescence parameters such as intensity, NPQ and QY using the measurements received from the sensor 530. The fluorescence parameters determined by the module 540 are used by a neural network control system 550. The neural network control system 550 implements a decision tree to adjust frequency and duty cycle of the LEDs 510 based on the determined fluorescence parameters. The adjusted parameters are transmitted to the generator 505 to adjust control of the LEDs 510.

In the example of FIG. 5, the LEDs 510 correspond to the at least one light source 405 of the assessment device 110 and the sensor 530 corresponds to the at least one sensor of the assessment device 110. The sensor 530 can in some arrangements include a sample plate (such as the sample plate 115) for receiving the sample of the fluid 515. The generator 505, module 540 and controller 550 correspond to the processor and controller of the assessment device 110.

The system and method for assessing optimised temporal variations of illumination may also be used within a photobioreactor. For example, in one embodiment, the assessment device 110 may be in communication with a photobioreactor, such that a sample of algae provided in the assessment device 110 is the algae being cultivated within the photobioreactor, and the assessment device 110 can determine the optimised temporal variations to be carried out by the photobioreactor. In further embodiments, the processor 310 of the assessment device 110 may be in communication with a processor of the photobioreactor, such that the light source of the photobioreactor may be controlled to provide the optimised temporal variations in line with the outputted optimised temporal variations of flux output conditions identified by the assessment device. In other embodiments, the assessment device 110 is incorporated into the photobioreactor, such that the light source of the photobioreactor is controlled to identify and provide temporal variations in flux output, and a closed loop feedback system is used to adjust the illumination to find the optimal temporal variations for the algae being cultivated in the photobioreactor. In this embodiment, the microplate 115 may be replaced with a tray which receives algae solution.

In further embodiments, a photobioreactor may be configured to carry out the determination of optimal temporal variations in flux output according to the present invention, in which, instead of providing an algae sample on a microplate, the algae is provided on a plurality of terraced illumination trays in a photobioreactor, and the light source is controlled to emit light towards the trays in accordance with flux output conditions. The photobioreactor may be provided with at least one sensor to monitor a response parameter, and determine and adjust the flux output conditions accordingly to provide a closed loop feedback system for improved algae growth. In another embodiment, existing photobioreactors such as a tubular photobioreactor, may be configured to carry out the determination of optimal temporal variations in luminous flux output according to the present invention.

### Performance Data

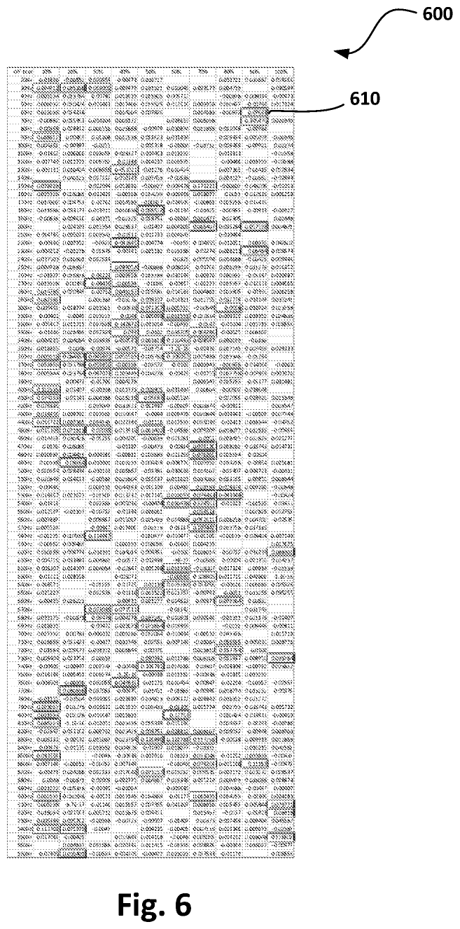

Tests of the assessment device 110 were run on a sample of Chlorella vulgaris. As shown in FIG. 6, there is a table 600 of the scan of frequency and duty cycles. The Green coloured values (identified as values highlighted by a thick border, such as 610) indicate higher photosynthetic efficiency in Chlorella vulgaris.

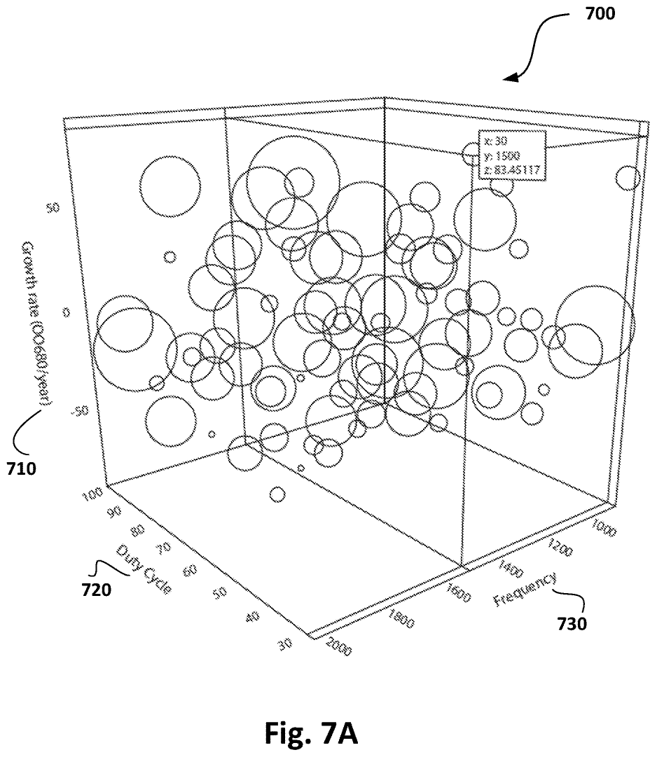

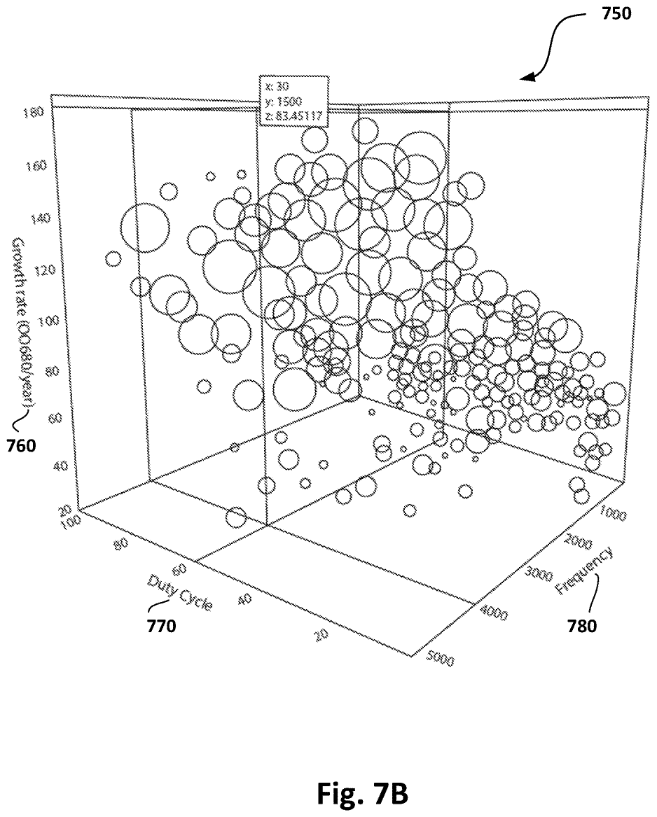

Using the assessment device 110, hundreds of different frequency and duty cycle combinations were scanned and certain flickering patterns were found that improve growth of algae. FIG. 7A shows a graph 700 of algae growth rate (710) compared to duty cycle (720) and frequency (730) from tests on a sample of Scenedesmus. FIG. 7B shows a graph 750 of algae growth rate (760) compared to duty cycle (770) and frequency (780) from tests on a sample of Chlorella vulgaris. As shown in FIGS. 7A and 7B, there was a discovery of improved growth when flickering illumination was provided at 30% duty cycle and 1500 Hz in Scenedesmus (FIG. 7A), and 60% duty and 4000 Hz in Chlorella vulgaris (FIG. 7B).

Although the invention has been described with reference to specific examples it will be appreciated by those skilled in the art that the invention may be embodied in many other forms.

1. A device for assessing temporal variations in flux output, including:
at least one light source able to emit light in accordance with temporally varying flux output conditions;
a sample plate affixed above the at least one light source, configured to receive at least one sample;
a processor in communication with the light source and configured to control the at least one light source to provide a flux output, the flux output including at least a duty cycle and a pulse frequency;
at least one sensor configured to obtain a measurement of at least one response parameter of the at least one sample; and
wherein the processor is further configured to control the at least one light source to adjust the provided flux output based on the at least one response parameter.

2. The device of claim 1, wherein the at least one light source and the sample plate are disposed on a modular base.

3. The device of claim 1 or claim 2, wherein the at least one light source is a light-emitting element.

4. The device according to claim 1, wherein the at least one light source is an array of light-emitting elements.

5. The device according to claim 1, wherein the flux output further includes intensity.

6. The device according to claim 1, wherein the sample is algae.

7. The device according to claim 6 wherein the algae is a microalgae.

8. The device according claim 1, wherein the duty cycle and the pulse frequency are adjusted to enable temporal variation of the flux output over a defined time interval.

9. The device according to claim 1, wherein the sample plate is a microplate.

10. The device according to claim 9, wherein the microplate is a single well microplate.

11. The device according to claim 9, wherein the microplate includes an array of equally spaced wells.

12. The device according to claim 9, wherein the microplate is a 96-well microplate.

13. The device according claim 1, wherein the at least one sensor is a CCD camera.

14. The device according to claim 13, wherein the CCD camera is configured to perform a fluorometric assessment.

15. The device according to claim 13 wherein the camera is fitted with a Chlorophyll a specific emission filter.

16. A method for determining optimal temporal variations in flux output for algal growth, including the steps of:
providing at least one sample on a sample plate;
controlling at least one light source to emit light towards the sample plate in accordance with flux output conditions, wherein the flux output conditions include at least a duty cycle and a pulse frequency;
exposing the sample to the emitted light for a predefined time interval;
obtaining a first measurement of at least one response parameter related to the at least one sample after it has been exposed to the emitted light for a defined time interval;
adjusting the flux output conditions based on the at least one response parameter;
obtaining a second measurement of at least one response parameter related to the at least one sample after it has been exposed to emitted light having adjusted flux output conditions for a second defined time interval;
identifying an optimal set of flux output conditions from the at least one response parameter when the response parameter is determined to reach a threshold value; and
outputting the optimal set of flux output conditions as an optimal temporal variation of flux output for the sample.

17. The method according to claim 16, wherein the first measurement or the second measurement is obtained by at least one sensor.

18. The method according to claim 16, wherein the at least one response parameter includes the results of a fluorometric assessment.

19. The method according to claim 16, wherein adjusting the flux output conditions includes adjusting at least one of the duty cycle or the pulse frequency.

20. The method according to claim 16, wherein identifying an optimal set of flux output conditions from the at least one response parameter includes comparing the first measurement and the second measurement.

21. The method according to claim 16, wherein identifying an optimal set of flux output conditions from the at least one response parameter incudes comparing the first measurement and the second measurement with historical measurements.
